(19) United States
(12) Patent Application Publication
(10) Pub. No.: US 2022/0192537 A1
(43) Pub. Date: Jun. 23, 2022

(54) CAPTURE AND DETECTION SYSTEM FOR SARS-COV-2 AND OTHER RESPIRATORY PATHOGENS

(71) Applicant: THE REGENTS OF THE UNIVERSITY OF CALIFORNIA, Oakland, CA (US)

(72) Inventors: Thomas Milner, Irvine, CA (US); Robert G. W. Brown, Irvine, CA (US); Nitesh Katta, Irvine, CA (US); Matthew Brenner, Irvine, CA (US); James Tunnell, Austin, TX (US); Mohadeseh Hashemidehagi, Austin, TX (US); Raj Nihalani, Irvine, CA (US); Wangcun Jia, Irvine, CA (US)

(21) Appl. No.: 17/554,794

(22) Filed: Dec. 17, 2021

Related U.S. Application Data
(60) Provisional application No. 63/127,850, filed on Dec. 18, 2020.

(51) Int. Cl. A61B 5/097; G01N 21/65; G01N 33/497; G01N 1/22; G06N 20/00; C12Q 1/686

(52) U.S. Cl. CPC A61B 5/097; G01N 21/658; G01N 33/497; G01N 1/22; B82Y 35/00; C12Q 1/686; G01N 2800/26; G01N 2001/2244; G06N 20/00

(57) ABSTRACT

The present invention features an optical detection system for SARS-CoV-2 or other pathogens, which includes a specialty mask. The specialty mask incorporates a SERS nanopatch for accumulating pathogenic particles from a wearers breath. When the SERS nanopatch receives incident NIR light, backscattered light from the SERS nanopatch is detected by a receiver and analyzed for a Raman spectral shift. Detection of the Raman spectral signature from the SERS nanopatch allows for determination if SARS-CoV-2 or another pathogen is present. In addition to the mask with a nanostructured surface for collecting pathogenic material, the system includes a laser source directed at the nanostructured surface, a detection system to collect backscattered light, a spectral analysis system to detect Raman shifted light, and an analysis system for determining if SARS-CoV-2 or another pathogen is present. AI image processing may be used to steer the laser beam safely to the nanopatch, avoiding eye contact.

### CROSS-REFERENCES TO RELATED APPLICATIONS

This application is a non-provisional and claims benefit of U.S. Provisional Application No. 63/127,850 filed Dec. 18, 2020, the specification(s) of which is/are incorporated herein in their entirety by reference.

### BACKGROUND OF THE INVENTION

### Field of the Invention

The present invention relates to a medical diagnostic detection device. More specifically, the present invention relates to a system for optical detection of SARS-CoV-2 and other respiratory pathogens in face-worn masks and other capture devices via surface-enhanced Raman spectroscopy (SERS).

### Background Art

SARS-CoV-2 and the ensuing COVID-19 pandemic have impacted the lives of most human beings living in almost every country on Earth. In the United States alone, the death of more than 178,000 individuals was attributed to a SARS-CoV-2 infection at the conclusion of summer 2020. Regions with a high population density such as New York City, New Jersey, and California have been more severely impacted than most rural areas. Some exceptions to the high population density criteria are states such as Texas that initially did not require mask-wearing and prematurely reopened public venues and soon witnessed a rapid increase in number of infections and COVID-19 deaths. After city and county mandates in Texas required mask-wearing and social-distancing, COVID-19 infection rates and deaths quickly declined.

The mental health consequences of the COVID-19 pandemic in the United States have been massive and devastating. Compared to 2019 data, the percentage of our population exhibiting symptoms of anxiety or depressive disorder has exploded. From the early days of the COVID-19 pandemic, the percentage of the US population exhibiting symptoms of anxiety or depressive disorder has hovered at about 30-35%—a two-hundred percent increase over the previous year. The widespread increase of anxiety and depression has exacerbated the social and economic wellbeing of many. Pre-existing frustrations on racial, social, and economic injustices have been amplified, resulting in widespread social unrest that has not been observed for generations. In early 2020, recognizing the potential for social and economic disruption, the US Congress and executive branch approved monthly stimulus payments to individuals most susceptible to economic displacement from COVID-19.

The COVID-19 pandemic has also seriously impacted the United States economy. The congressional budget office has projected that the Federal Governments deficit in 2021 alone will be $2.77 trillion. Some economists have voiced concern that without prudent and careful management such large increases in Federal debt may compound risks of further economic and social instability. Unfortunately, the economic burden has been heaviest for those individuals amongst us who are least equipped to deal with the impact. Exacerbating these concerns is a recognition that unless we prepare our communities now for future pandemics or possible SARS-CoV-2 reinfections, similar or even more severe consequences than those experienced with COVID-19 may occur.

Underlying the challenge of managing a pandemic is the relationship between ensuring individual health vs. societal economic wellbeing. Although extreme social isolation is possibly the most effective measure to mitigate against SARS-CoV-2 transmission, impact on mental health for many is severe and dangerous. Moreover, social isolation also negatively impacts the economy by disrupting many businesses that require frequent and close social interactions (e.g. restaurants, theaters, and fitness facilities). On the other hand, absence of physical barriers between individuals rapidly increases chances of SARS-CoV-2 transmission and endangers the lives of many who are most vulnerable to infection (e.g., the immune-compromised and elderly). Tragically, numerous recent examples of both extremes have been observed.

Along with the widespread distribution of SARS-CoV-2 vaccines and boosters as well as constant monitoring of variants (e.g. Delta, Omicron), the most effective strategies to maintain sustainable economic activity while mitigating against individual transmission is adoption of a required mask-wearing policy and implementation of widespread, rapid, cost-effective and accurate SARS-CoV-2 screening. Although masks do not entirely prevent transmission of SARS-CoV-2 they significantly reduce the number, concentration, and ‘reach’ of airborne viral particles emitted from an infected individual—decreasing the viral load. A widespread, rapid, cost-effective, and accurate SARS-CoV-2 screening approach is also an essential component for pandemic management as many younger individuals are asymptomatic and without early detection, SARS-CoV-2 can spread rapidly through a community in an exponential manner endangering the lives of the most vulnerable individuals.

There is a need for rapid, remote (e.g. non-contact), high-throughput detection of SARS-CoV-2, without tissue harvest. Current solutions, such as molecular diagnostic tests, antigen diagnostic tests, and viral antibody tests, while having made strides in speed and accuracy over the last year, still struggle with speed, cost, and relative accuracy.

### BRIEF SUMMARY OF THE INVENTION

It is an objective of the present invention to provide systems, devices, and methods that allow for optical detection of SARS-CoV-2 and other respiratory pathogens, as specified in the independent claims. Embodiments of the invention are given in the dependent claims. Embodiments of the present invention can be freely combined with each other if they are not mutually exclusive.

The present invention provides a solution that works via multiple mechanisms. For example, the present invention may feature an intelligent mask that mitigates against individual transmission, while simultaneously allowing for widespread, extremely-rapid, low-cost, repeatable, and accurate SARS-CoV-2 screening. The COBRA Kiosk provides a system for COVID-19 Breath and Respiratory Analysis. The COBRA Kiosk system may include a mask-embedded SERS (Surface Enhanced Raman Spectroscopy) nanopatch for automatic at-a-distance SARS-CoV-2 screening.

The screening system of the present invention is fast, inexpensive, and scalable to perform tens of millions of screenings each day. Even if the specificity of the SERS nanopatch screening were relatively low (e.g., 80%) and some secondary testing with a more accurate conventional test (e.g., PCR) was required, the time and cost savings of the system of the present invention are substantial. Importantly, the massive scaling potential and associated high screening rates of the screening and intelligent mask system of the present invention provides a valuable and effective tool for COVID-19 pandemic management.

One of the unique and inventive technical features of the present invention is the inclusion of a SERS nanostructure in an antiviral facial mask. Without wishing to limit the invention to any theory or mechanism, it is believed that the technical feature of the present invention advantageously provides for optical screening of an individual wearing the mask, via SERS. None of the presently known prior references or work has the unique inventive technical feature of the present invention.

Existing SARS-CoV-2 testing approaches have a number of immutable characteristics that seriously limit their utility to serve as an effective screening tool to manage the COVID-19 pandemic. Many of these limitations originate in the increased cost and extended times to complete tests, with results that are often inaccurate and highly variable. A key factor in the increased cost, extended screening times, and high uncertainty of existing techniques is the requisite manual collection of bodily fluids and/or tissue material by a human operator. The increased cost and extended testing times restrict the effective screening rate and severely limit capability to identify asymptomatic individuals and control the pandemic through quarantining. For example, if a screening test is administered weekly and an asymptomatic youth contracts SARS-CoV-2, interactions with friends and family members over ensuing days can infect numerous people. The following three innovations of the COBRA Kiosk screening system of the present invention resolve limitations of existing tests.

Innovation 1: No manual collection of bodily fluids and/or tissue material. An important innovation of our COBRA Kiosk screening system is that manual harvest of bodily fluids and/or tissue material by a human operator is unnecessary. By replacing human tissue harvest with precise, safe, and controlled at-a-distance optical sampling, the inaccuracy and large variability associated with existing tests may be substantially reduced. The mask-embedded SERS nanopatch of the present invention collects SARS-CoV-2 viral particles that the test subject may be infected with, without the need for manual collection of bodily fluids or tissue material. Moreover, because the intelligent mask is positioned over the oral and nasal cavities for extended time periods (e.g. hours), mass of collected bodily fluids and associated materials accumulate on the mask-embedded SERS nanopatch thereby increasing accuracy of subsequent COBRA screenings.

Innovation 2: SERS nanopatch allows rapid, low-cost, and accurate SARS-CoV-2 screening. In contrast with existing single-use testing techniques, optical screening is non-contact, non-destructive, and allows for repeated screenings over extended periods of time. SERS or Surface Enhanced Raman Spectroscopy can enhance a specimen's Raman spectral signal amplitude by many orders of magnitude. Because the cost to generate and detect photons and complete a SERS measurement is relatively low, optical screening is cost-effective and fast. Using nanostructured surfaces. SERS may be applied for the accurate detection of multiple virus types.

Innovation 3: Economical COBRA screening. Relatively low incidence of SARS-CoV-2 infections in most populations (e.g., 1% or less) suggests that a candidate screening approach should have high sensitivity. Importantly, since testing is preferably applied uniformly to all individuals at a given venue, time and cost savings of a COBRA screening is relatively inelastic to the tests specificity. To illustrate, to examine 1000 individuals, a six-second COBRA screening that has 98% sensitivity and just 80% specificity compares very favorably with a conventional three-minute test costing $5 (Table 1).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The patent application or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A shows an illustration of the function of the SERS nanopatch of the present invention for SARS-CoV-2 detection. SERS nanopatches are fabricated with embedded SERS active nanomaterials and affixed to the outer surface of the mask where SARS-CoV-2 accumulates within the nanopatch. When illuminated with near-infrared (NIR) laser light, the SERS nanomaterial enhances the Raman molecular fingerprint spectrum of the virus, which is recorded. Machine learning models then classify the SERS fingerprint spectrum as positive or negative for SARS-CoV-2.

FIG. 1B shows a table or time and cost savings of a six-second COBRA screening for 1000 individuals.

FIGS. 2A-2C show results demonstrating SERS detection of SARS-CoV-2. FIG. 2A shows photographs of a custom-built Raman system with a hand-held fiber optic probe for SERS measurement. FIG. 2B shows an absorbance spectrum of gold nanostars (AuNS) with peak absorption in the NIR. FIG. 2C shows Raman spectra of various samples and controls, demonstrating strong SERS enhancement within the SARS-CoV-2 sample when AuNS are present.

FIG. 3 shows Raman spectra of common mask materials for evaluation as nanopatch substrates.

FIG. 4 shows an illustration of the experimental setup of an aerosolized SARS-CoV-2 simulator. Virus is aerosolized using a nebulizer and the air flow is controlled via a fan. Nanopatches may be calibrated for viral density as a function of viral load, air flow rate, and exposure time.

FIG. 5 shows a schematic of a Raman laser-scanning bench-top system for mask measurements. DM: dichroic mirror; GM: galvanometer mirror; RC: reflective collimator; GTL; Galilean beam expander, OL: objective lens with a working distance of 30 cm.

FIG. 6A shows a front view of the COBRA Kiosk displaying the Intelligent mask alignment system feedback to the user and guiding the placement of the mask in the SERS field of view.

FIG. 6B shows a 3D rendering of the Kiosk.

FIG. 6C shows an illustration of the optical setup of the visible camera, SERS and IR camera.

FIG. 6D shows an illustration of a subject in front of the kiosk being screened.

FIG. 7 shows an illustration of a facemask with trackers and bio-sensors positioned on the face of a user.

FIG. 8 shows an illustration of the layers and components of a facemask with silver nanoparticle impregnated layers.

FIG. 9 shows an illustration of a user being screened by the laser detection system of a kiosk of the present invention. In this example, the user is additionally provided with hand sanitizer by a sanitizer dispenser.

FIG. 10A shows a schematic illustration of an optical circuit for low-frequency Raman detection.

FIG. 10B shows another schematic illustration of an optical circuit for low-frequency Raman detection.

FIG. 10C shows a schematic illustration of low-frequency Raman detection, in which optical heterodyning combined with optical homodyne detection (lock-in amplifier) allows ultra-high sensitivity for detection.

FIG. 11 shows a table of characteristics of various laser sources for low-frequency Raman sensing.

FIG. 12 shows a graph comparing detection of Rhodamine B between a reference signal, a low concentration on paper, and a low concentration on the nanopatch of the presently claimed invention.

FIG. 13A-13B shows Raman spectral graphs comparing detection of heat-inactivated SARS-CoV-2 between using the nanopatch (A) and a silicon substrate (B) to measure the capabilities of the nanopatch of the present invention.

### DETAILED DESCRIPTION OF THE INVENTION

The COBRA Kiosk system of the present invention allows for high-throughput, scalable and economical SARS-CoV-2 screening. The system may operate by first detecting the presence of an individual who is facing the COBRA Kiosk, a handheld detector device, etc. and is wearing an intelligent mask with an embedded SERS nanopatch. After a confirmed presence, the COBRA Kiosk screening system may provide visual feedback to the individual to aid facial self-positioning. After the intelligent camera system verifies an image-lock on SERS nanopatch fiducials, the COBRA Kiosk screening system may perform a rapid sequence of SERS nanopatch measurements (e.g. intensity). To ensure safe operation, prior to recording each SERS nanopatch measurement, an image-lock on nanopatch fiducials may be first verified. Recorded SERS data may be input into a decision-making algorithm that determines if the test subject is SARS-CoV-2 negative or may require a secondary high-accuracy test (e.g., PCR). The decision-making algorithm may comprise a machine learning algorithm trained by a set of data of intensities mapped to the presence of SARS-CoV-2 or other diseases. The intelligent mask may be 25 cm to 40 cm in width and 8 cm to 17 cm in height.

High detection sensitivity of SARS-CoV-2 is fundamental to realizing the benefits of the COBRA Kiosk screening system. Inasmuch as the SARS-CoV-2 infection rate in most demographics is low (e.g., 1%), an important societal benefit of the COBRA Kiosk is a substantial reduction in number of individuals who must undergo more expensive and longer duration testing. A second challenge for the COBRA Kiosk SARS-CoV-2 screening system is mis-direction of SERS excitation light. Mis-directed SERS excitation light can arise from two scenarios. First, apparent direction of SERS excitation light (i.e., at the nanopatch) may be different from the actual beam intersection site on the test subject. Second, even when SERS excitation light is correctly directed at the mask-embedded nanopatch, scattered light from airborne particulates in the beam path may present a risk. The present invention includes approaches to mitigate each risk. A third challenge for successful use of the COBRA Kiosk screening system is the enormous variation of expected human kinetic movements when approaching and standing for a SARS-CoV-2 screening. Compensation for kinematic movements while standing for a COBRA screening must be used to ensure an image-lock on nanopatch fiducials is verified so that the SERS excitation beam is safely directed at the nanopatch for each acquisition. To ensure safe, accurate, and repeatable beam aiming at the mask-embedded SERS nanopatch, a wide range of human kinematic movements may be tested. Finally, a fourth challenge is the capability of the decision-making algorithm to determine whether a test subject is SARS-CoV-2 negative or alternatively may require a secondary higher-accuracy test. The decision-making algorithm may be developed and iterated throughout the entire program development life-cycle. Careful selection of SERS nanostructured surfaces and their associated signal enhancements may be considered to realize a robust algorithmic approach. Extensive algorithm development using retrospectively recorded COBRA kiosk screening data from robot-actuated mask-wearing dummies may be completed. Successful COBRA Kiosk operation may be confirmed by testing the decision-making algorithm using ‘live’ mask-wearing dummies that simulate actual in-person screenings.

Talati and Jha have reported on acoustic phonon quantization and low-frequency Raman spectra of spherical viruses and showed variations of low-frequency Raman peaks with the size of a spherical virus. At 1 μm, the 0.1 cm¹ shift is about 3 GHz. (Physical Review E 73. 011901 (2006)) Park and Lee have reported on development of CNT-metal-filters by direct growth of carbon nanotubes, and Dresselhaus et. al. have reported on Raman spectroscopy of carbon nanotubes. Luo et. al. have published a review on nanofabricated SERS-active substrates for single-molecule to virus detection in vitro.

In some embodiments, the present invention features a system for high-throughput pathogenic particle screening. As a non-limiting example, the system may include: a facemask for capturing and preparing pathogenic particles for screening, and a surface-enhanced Raman spectroscopy (SERS) detector, configured to record a SERS spectrum from the facemask or capture device, so as to provide for high-throughput screening for the pathogenic particle. In some embodiments, the facemask may include: a barrier material, configured to allow air flow through the barrier material and to at least partially block the passage of pathogenic particles through the barrier material: and a nanostructured material, configured to enhance a Raman scattering signal amplitude of the pathogenic particles. In preferred embodiments, the facemask may be designed to be positioned over the oral and nasal cavities of a user so as to capture any particles expelled by the user on the nanostructured material, and to prepare the particles for screening.

The nanostructured material may be a material with structures with at least one dimension less than 100 nm. Without wishing to limit the present invention to any particular theory or mechanism, it is believed that these nanostructures allow for surface interactions with pathogenic particles that aid in their detection, for example, via the generation of surface plasmons. In some embodiments, the nanostructured material may be replaced with a non-nanostructured plasmonic structure.

In other embodiments, the system may use particle collection substrates other than facemasks to support the nanostructured material and to collect the particles for screening. As a non-limiting example, a swab (e.g. a nasal or oral swab) may be used for mechanical collection of particles for screening. As another non-limiting example, an air filter such as an airplane cabin filter or a building HVAC filter may be used to collect airborne particles for screening. In some embodiments, the particles may be captured directly on a nanostructured material. In other embodiments, the particles may be captured and then transferred to a nanostructured material for screening. Robotic devices may be used to aid in the efficiency of particle collection and screening. For example, a robotic device may assist in the harvesting of biomaterial, the transfer of harvested biomaterial onto a nanostructured material, the positioning of the nanostructured material relative to a detector, or other tasks involved in the screening process.

The facemasks or other particle collection substrates may be disposable or reusable. They may be designed for either short-term or long-term use. As non-limiting examples, the facemasks or other particle collection substrates may be designed to be used for a duration of minutes, hours, or days. In some embodiments, the nanostructured material may be able to be cleaned so as to extend the effective lifetime of the particle collection substrate.

The present invention may be used for the detection of various respiratory pathogens such as viruses and bacteria. While one especially relevant current example is SARS-CoV-2, both RNA and DNA-based viruses may be detected using the present invention. As a non-limiting example, the pathogenic particle may be a SARS-CoV-2, Coronavirus, middle east respiratory syndrome (MERS), severe acute respiratory syndrome (SARS) coronavirus, influenza, zika virus, Herpes, Zoster, Flavivirus, Redondo virus, Orthomyxovirus, Picornavirus, Papillomavirus, Syncytial virus, Adenovirus, human immunodeficiency virus (HIV), Circovirus, Anellovirus, Polyoma virus, Cytomegalovirus, Variola virus, Epstein-Barr virus, bacteria-invading virus, influenza, measles, mumps, rhinovirus, pertussis, or tuberculosis (TB) particle. The particles that may be detected using the present invention range in size from less than 20 nm to over 100 μm.

In some embodiments, the detector may include a laser light source, such as an infrared, visible, or ultraviolet laser light source. In some embodiments, an ultraviolet laser light source may provide information on DNA or RNA. In some embodiments, the laser light may be directed at a portion of the facemask or at another substrate for the nanostructured material. The system may additionally include a microprocessor configured to classify the SERS spectrum as positive or negative for the pathogenic particle. In some embodiments, the microprocessor may use a machine-learning algorithm to classify the SERS spectrum. In some embodiments, the SERS detector uses optical heterodyning for ultra-low frequency Raman spectroscopy. The ultra-low frequency Raman spectroscopy may be configured for the 10 GHz range, or for the 5-20 GHz range.

In preferred embodiments, the SERS detector is a remote detector and the facemask may be screened for the pathogenic particle at a distance. The system may additionally include a second test such as a polymerase chain reaction (PCR) test or a viral antibody test. In some embodiments, the SERS detector may be housed in a kiosk. The kiosk may additionally include a visible-wavelength camera and an infrared wavelength camera, and may be configured to detect a position of the facemask in relation to the SERS detector. In some embodiments, the kiosk may be configured to provide visual and/or audible feedback to the user so as to guide placement of the facemask within a field of view of the SERS detector.

In other embodiments, the detector may be a handheld detector. As a non-limiting example, the handheld detector may include a fiber optic probe. In some embodiments, the system does not require collection of bodily fluids or tissue material. In other embodiments, the system uses collection of bodily fluids or tissue material. In preferred embodiments, the system allows for multiple screenings over an extended period of time. In some embodiments, the SERS detector is integrated into a body scanner such as an airport security scanner. The SERS detector may be mounted on a component of the body scanner which rotates around the user.

In some embodiments, the present invention features a facemask for capturing and preparing a pathogenic particle for screening. As a non-limiting example, the facemask may include: a barrier material, configured to allow air flow through the barrier material and to at least partially block the passage of pathogenic particles through the barrier material; and a nanostructured material, configured to enhance a Raman scattering signal amplitude of the pathogenic particle. The facemask may be configured to be positioned over the oral and nasal cavities of a user so as to capture any pathogenic particles expelled by the user on the nanostructured material, so as to prepare the particle for screening.

In some embodiments, the nanostructured material comprises a nano-patch. In other embodiments, the nanostructured material comprises a nanosurface or a dispersion of nanoparticles, nano-rods, nano-stars, nano-spheres, nano-cylinders, nano-cubes, nano-ellipsoids, or nano-planar or nano-spiral-twisted particles. Other nano-shapes which exhibit sufficiently strong nano-plasmonic and/or nano-polaritonic properties may also be used. Such particles may be spatially-arrayed randomly or in geometric-coupled-arrays for enhanced Raman-signal amplification. The nanostructured material may include gold, silver, copper, aluminum, another metal, or a doped semiconductor (e.g. a heavily doped semiconductor).

In some embodiments, the facemask may additionally include one or more CO2 sensors. The CO2 sensors may be configured to change color when the facemask has been worn for a sufficient length of time for accurate screening. In some embodiments, the facemask may additionally include a quick response (QR) code linked to an identifier for the user.

The present invention features a system for high-throughput pathogenic particle screening. In some embodiments, the system may comprise a facemask for capturing and preparing pathogenic particles for screening. The facemask may comprise a barrier material, configured to allow air flow through the barrier material and to at least partially block the passage of pathogenic particles through the barrier material. The facemask may further comprise a nanostructured material, configured to enhance a Raman scattering signal amplitude of the pathogenic particles. The facemask may be configured to be positioned over the oral and nasal cavities of a user so as to capture any particles expelled by the user on the nanostructured material, so as to prepare the particles for screening. The system may further comprise a surface enhanced Raman scattering (SERS) detector, configured to record a SERS spectrum from the facemask, so as to provide for high-throughput screening for the pathogenic particle.

The pathogenic particle may be a SARS-CoV-2, Coronavirus, middle east respiratory syndrome (MERS), severe acute respiratory syndrome (SARS) coronavirus, influenza, zika virus, Herpes, Zoster, Flavivirus, Redondo virus, Orthomyxovirus, Picornavirus, Papillomavirus, Syncytial virus, Adenovirus, human immunodeficiency virus (HIV), Circovirus, Anellovirus, Polyoma virus, Cytomegalovirus, Variola virus, Epstein-Barr virus, bacteria-invading virus, influenza, measles, mumps, rhinovirus, pertussis, or tuberculosis (TB) particle. The detector may include an laser light source, configured to be directed at a portion of the facemask. The system may further comprise a microprocessor configured to classify the SERS spectrum as positive or negative for the pathogenic particle through a machine learning algorithm. The SERS detector may use optical heterodyning for ultra-low frequency Raman spectroscopy configured for the 0.1-50 GHz range. The SERS detector may be a remote detector and the facemask may be screened for the pathogenic particle at a distance. The second test may comprise a polymerase chain reaction (PCR) test or a viral antibody test. The SERS detector may be housed in a kiosk comprising a visible wavelength camera and an infrared wavelength camera for detecting a position of the facemask in relation to the SERS detector. The kiosk may be configured to provide feedback to the user so as to guide placement of the facemask within a field of view of the SERS detector. The detector may be a handheld fiber optic probe. Use of the system may not require collection of bodily fluids or tissue material. The SERS detector may be mounted on a component of a body scanner which rotates around the user.

The present invention features a facemask for capturing and preparing a pathogenic particle for screening. In some embodiments, the facemask may comprise a barrier material, configured to allow air flow through the barrier material and to at least partially block the passage of pathogenic particles through the barrier material. The facemask may further comprise a nanostructured material, configured to enhance a Raman scattering signal amplitude of the pathogenic particle. The facemask may be configured to be positioned over the oral and nasal cavities of a user so as to capture any pathogenic particles expelled by the user on the nanostructured material, so as to prepare the particle for screening.

The nanostructured material may comprise a nano-patch, a nanosurface or a dispersion of nanoparticles, nano-rods, nano-stars, nano-spheres, nano-cylinders, nano-cubes, nano-ellipsoids, nano-planar or nano-spiral-twisted particles, gold, silver, copper, aluminum, another metal, or a doped semiconductor. The facemask may further comprise one or more CO2 sensors. The CO2 sensors may be configured to change color when the facemask has been worn for a sufficient length of time for accurate screening. The facemask may further comprise a quick response (QR) code linked to an identifier for the user.

The present invention features a system for high-throughput pathogenic particle screening. In some embodiments, the system may comprise a capture device for capturing and preparing pathogenic particles for screening. The capture device may comprise a nanostructured material, configured to enhance a Raman scattering signal amplitude of the pathogenic particles. The capture device may further comprise a support structure for supporting the nanostructured material. The capture device may be configured to capture particles from the user on the nanostructured material, so as to prepare the particles for screening. The system may further comprise a surface enhanced Raman scattering (SERS) detector, configured to record a SERS spectrum from the capture device, so as to provide for high-throughput screening for the pathogenic particle. The capture device may further comprise a barrier material configured to allow air flow through the barrier material and to at least partially block the passage of pathogenic particles through the barrier material. The capture device may comprise a swab, an air filter, or other test structure.

### Example

The following is a non-limiting example of the present invention. It is to be understood that said example is not intended to limit the present invention in any way. Equivalents or substitutes are within the scope of the present invention.

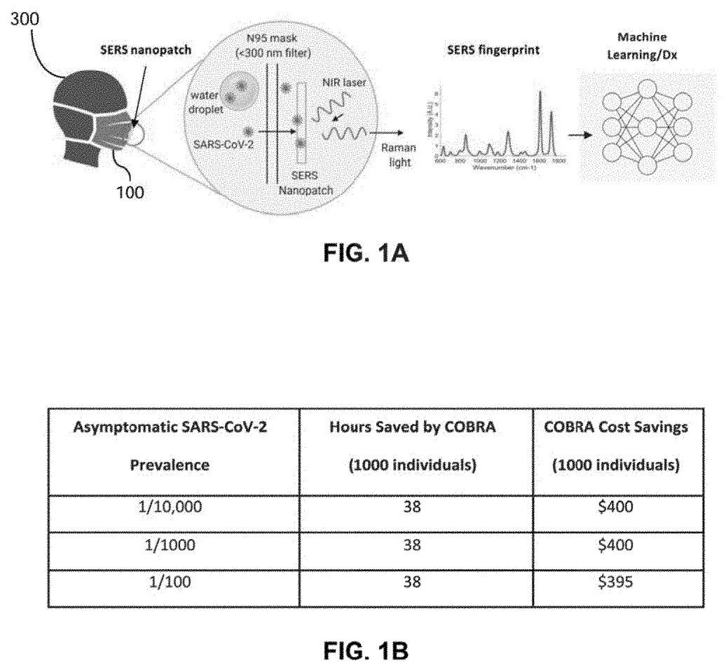

This example utilizes surface-enhanced Raman scattering (SERS) to detect SARS-CoV-2 within the mask (FIG. 1A). SERS is a laser-based sensing technique that utilizes light scattered from a sample to determine its molecular composition. Traditional, un-enhanced Raman spectroscopy has been used for decades to interrogate material composition and is sensitive to the material's vibrational molecular structure. In biological samples, Raman spectroscopy is sensitive to the composition of lipids, proteins, nucleic acids, and amino acids and can be used as a so-called “molecular fingerprint” to identify contaminants, bacteria, and viruses. However, the Raman effect is weak, severely limiting its speed and accuracy as a low-concentration bioanalytical sensor. SERS enhances a sample's Raman signal by many orders of magnitude using nanomaterials placed in close proximity to the analyte of interest. The same laser light used to measure the Raman scattering signal is used to resonate electrons in metallic nanostructures (known as plasmon resonances) that create very high local electromagnetic fields that have been shown to enhance the Raman signal by as much as eight orders of magnitude (i.e., 10). SERS has been shown to provide ultrasensitive detection with single molecule sensitivity, and it has been used to detect bacteria and viruses. Recent efforts have shown detection of respiratory viruses (influenza, parainfluenza, rhinovirus) in clinical samples with detection sensitivity on par with RT-qPCR (10² EID₅₀/μL) at specificities of 90%.

This example demonstrates the use of “SERS nanopatches” for the detection of SARS-CoV-2 at a limit of detection at the level of current RT-qPCR test (10² copies/mL). Candidate SERS nanomaterials are first screened for optimal detection in terms of minimum detectable viral load. Optimal nanomaterials are then used to fabricate SERS nanopatches, and their accuracy (sensitivity and specificity) is assessed using an aerosolized SARS-CoV-2 simulator.

### Studies Demonstrating SERS Detection of SARS-CoV-2.

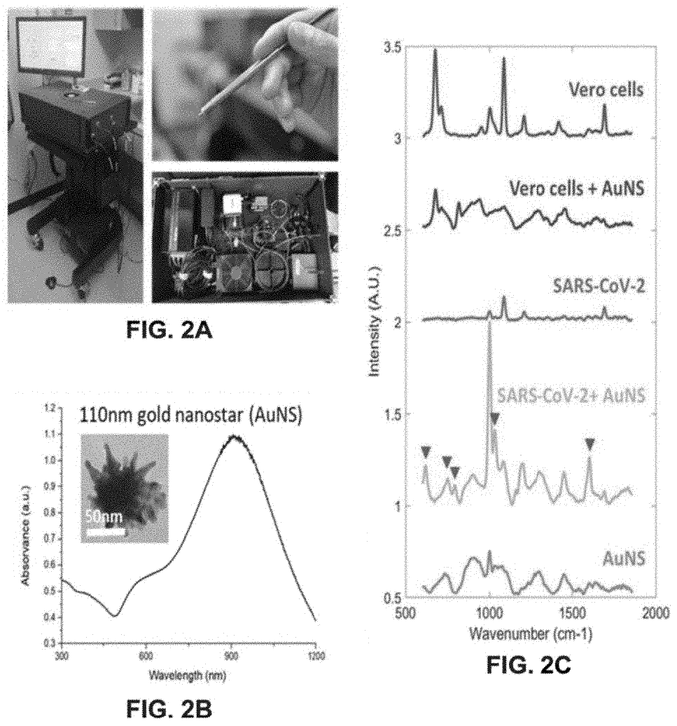

Results demonstrate surface enhanced Raman spectra (SERS) from SARS-CoV-2 with uniquely identifiable bands that are significantly enhanced over non-enhanced Raman (i.e., Raman of the virus alone). A clinical system is employed that utilizes a handheld, fiber optic probe to acquire Raman fingerprint spectra (600-1900 cm⁻¹) using near-infrared (NIR) excitation at 830 nm (FIG. 2A). In the present example, gold nanostars (AuNS) are employed as the SERS nanomaterial. The gold nanostars were prepared by using the seed-mediated growth method, resulting in an average diameter of 110 nm and a plasmon resonance between 700-1000 nm (FIG. 2B), aligned with a 830 nm Raman excitation source.

The examples shown in FIG. 2C demonstrate surface-enhanced Raman spectra (SERS) from SARS-CoV-2 with uniquely identifiable bands that are significantly enhanced over non-enhanced Raman (i.e., Raman of the virus alone). Raman spectra were acquired from SARS-CoV-2 samples with and without nanostars as well as from controls that included Kidney Vero-E6 cells (with and without nanostars) and nanostars alone. The Vero-E6 cells were chosen as a control because the SARS-CoV-2 samples contain Vero-E6 cell lysate as part of the culture process. 20 μl of Kidney Vero-E6 and heat-inactivated SARS-CoV-2 were dropwise added to the 20 μl (3 μg/ml) of gold nanostar and stirred for 5 min. Then, 20 μl of the mixture was pipetted on a coverslip (MgFl) and Raman spectra were immediately acquired using the fiber probe. Importantly, each of these spectra were acquired within 3 seconds.

Strong SERS enhanced Raman spectral bands were observed in SARS-CoV-2 samples with gold nanostars that were unique compared to either SARS-CoV-2 alone, Vero-E6 cells alone, or nanostars alone (FIG. 2C). Most bands observed in the gold nanostar alone samples result from the background in the Raman probe itself and are not associated with the sample. Note several key observations. First, the spectral features observed in the virus alone sample are also observed in the Vero-E6 cell sample, indicating that the unenhanced Raman spectrum of the viral sample does not show a strong independent viral signal fingerprint. Second, the SERS spectrum of the SARS-CoV-2 (sample with the nanostars) shows a number of additional large amplitude Raman bands not present in the Vero-E6 (with or without nanostars) or SARS-CoV-2 unenhanced spectrum (see red arrows in FIG. 2C). These additional peaks appear to be specific to the SERS SARS-CoV-2 sample. Finally, note the high signal-to-noise ratio of these SERS spectra were recorded with only a three second integration time, suggesting sufficient signal may be obtained in the COBRA Kiosk of the present invention. Additional controls and experiments elucidate the details of these spectra, enhancement factors, and optimum performance of SARS-CoV-2 detection.

Screening candidate SERS nanopatches and validating SARS-CoV-2 detection accuracy: The goal is to screen candidate SERS nanomaterials for optimal detection of SARS-CoV-2 and determine the performance (sensitivity and specificity) of SARS-CoV-2 detection using an aerosolized SARS-CoV-2 simulator.

Screen Candidate SERS Nanopatch Materials for SARS-CoV-2 Detection.

The minimum detection limits of SARS-CoV-2 across at least five candidate nanopatch materials are determined. Each of the candidate nanopatch materials have been shown to produce SERS enhancements; however, the optimum enhancement for a given material depends on interaction and attachment to the SARS-CoV-2 viral target. In addition, the cost and fabrication protocols of these materials vary widely. Therefore, determination of which nanopatch materials are candidates for SARS-CoV-2 detection guides selection of materials. Nanopatch materials may be considered candidates if they allow minimum detection of SARS-CoV-2 of at least 10² copies/mL—the detection limit of RT-qPCR and recent demonstrations of SERS detection of influenza.

The following four candidate nanoparticles have been chosen: silver nanoparticles, silver nanorods, gold nanorods, and gold nanostars. Silver nanoparticles represent a low-cost and facile method to produce a SERS substrate with a size of approximately 5 nm which closely mimics the type of nanosilver being used in current antimicrobial masks. Silver and gold nanorods provide enhanced near-infrared (NIR) plasmon resonances and allow the use of NIR laser excitation and higher light fluence safety limits for eye exposure. Gold nanostars have been reported to have some of the highest SERS efficiencies with the largest enhancement areas surrounding the material, thus representing an ideal substrate in terms of highest signal-to-noise ratio, albeit at a potentially higher cost.

A serial dilution experiment is performed for each nanomaterial using cultured, deactivated SARS-CoV-2 virus (VR-1986HK™). Solutions of nanoparticle spiked with order of magnitude dilutions decreasing from 104 to 10 copies/mL of SARS-CoV-2 virus are measured. Nanoparticle concentrations are varied from 10⁶ to 10¹⁰ particles/mL. Samples are repeated in replicates of five. Raman spectra are measured from each sample using a custom-built confocal Raman microscope. Following the protocol of Terrones et al., up to one hundred spectra are recorded for each sample to build a high-quality database of fingerprint spectra for SARS-CoV-2. Negative controls include viral media (supernatant of the centrifuged virus for purification), Vero-E6 cells, several strands of avian influenza A virus (H5N2, H7N2), rhinovirus, and parainfluenza (HPIV3). The SERS nanomaterial and virus interaction is confirmed using TEM and SEM.

The outcomes of this task include a high-quality SERS fingerprint database of SARS-CoV-2 and the control respiratory viruses and the minimum detectable viral loads of each of the candidate SERS nanomaterials. Materials are considered candidates if they detect viral loads below 10² copies/mL.

Characterize SERS Nanopatch Detection Accuracy Using Aerosolized SARS-CoV-2.

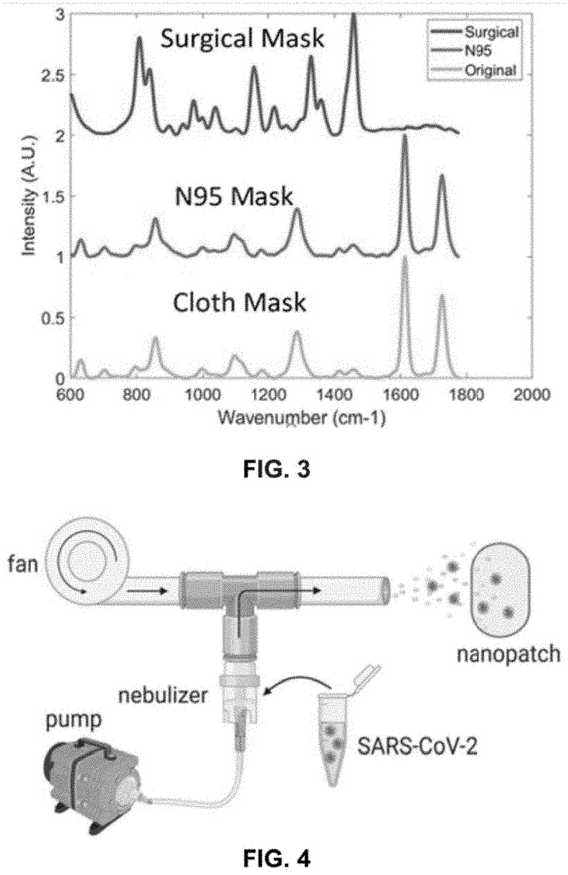

One candidate nanomaterial is chosen to fabricate SERS nanopatches. Nanopatches are fabricated by doping patch substrates (˜1 cm²) with the SERS nanopatch material. A nanopatch substrate from candidate materials with low Raman backgrounds is selected. Then, the accuracy (sensitivity and specificity) of SARS-CoV-2 detection is characterized using an aerosolized SARS-CoV-2 simulator. The nanopatch substrate is chosen from a review of Raman features of several common substrates used to fabricate masks and fabrics. An ideal material would have a low intrinsic Raman fingerprint in the spectral regions where the SARS-CoV-2 Raman fingerprints are the strongest. This would minimize interference with the SARS-CoV-2 detection. An initial review (FIG. 3) shows an array of Raman fingerprints for common mask substrates used for standard surgical masks (polypropylene) and industrial N95 masks and fabrics (polyester). Nylon and cotton have also shown to be suitable SERS substrates.

Once a substrate is chosen, the accuracy (sensitivity and specificity) of SARS-CoV-2 detection is characterized using an aerosolized SARS-CoV-2 simulator (FIG. 4). While viral loads in fluid samples can be characterized in terms of virus copies per unit fluid volume, detectable viral loads on masks and their relationship to detectable viral loads in bodily fluids were recently unknown. A metric for aerosolized virus that will be captured using the mask SERS nanopatch may require a novel metric of viral load. SARS-CoV-2 virus from culture is nebulized from solutions at known concentrations (confirmed by TEM and optical density via stained sample) and inserted into a flow stream aimed at nanopatch samples. Nanopatches are assessed for viral density using TEM. In order to calibrate the simulator, the nebulizer solution is spiked with known concentrations of virus (order of magnitude increments of viral load from 10-10⁵ copies/mL) and the deposited virus on the nanopatch is monitored using TEM. This results in a calibrated aerosolized viral load simulator with viral density on the mask calibrated to fluid volume viral load. This simulator is then used to characterize the accuracy of SERS nanopatches in detection of SARS-CoV-2 for various viral loads (low: 10² copies/mL, medium: 104 copies/mL; high: 10⁶ copies/mL). Negative controls include nebulized culture media without virus and inactivated respiratory viruses (influenza A).

Raman spectra are used to generate a receiver operating characteristic (ROC) curve for the detection of SARS-CoV-2. Spectra are preprocessed to calibrate for wavenumber, remove cosmic rays, and remove fluorescent and dark current backgrounds. Principal component analysis (PCA) is used to reduce to dimensionality of the spectra and utilize the scores for a logistic regression analysis to produce a ROC curve. In addition, an array of standard machine learning algorithms (support vector machine, decision tree, and random forest) are explored to classify the spectra. A 3-fold cross-validation is used, whereby the dataset is split into three equal groups, one group is used for the validation set and the other groups used as the training set. The average accuracy of the validation is used to select the best model. The area under the curve (AUC) and specificity at a fixed sensitivity of 98% are used as performance metrics of accuracy. The recent approach of Terrones (PNAS 2020) is followed to estimate sample size of one hundred.

Outcomes:

SARS-CoV-2 SERS fingerprint; candidate SERS nanomaterials and substrates for nanopatch fabrication; and accuracy for SARS-CoV-2 detection are determined. Milestones: Detection of SARS-COV-2 at less than 10² copies/mL; 98% sensitivity and 80% specificity.

Alternative Approaches:

SERS nanoparticles may be used for seeding the nanopatch substrates. Other hybrid particles (Au—Ag) and nanostructured surfaces that can allow for increased control and repeatability of the sample may also be used. In addition, methods may be used to enhance viral proximity to the nanomaterials using charge or antibody attachment or enrichment using substrates.

Design, construction and validation of COBRA kiosk: A mask-scanning COBRA kiosk is realized that: 1) automatically detects presence of a human subject wearing an intelligent mask with an embedded SERS nanopatch; and 2) safely records a SERS nano-patch measurement. A decision-making algorithm is developed for determining whether the test subject is required to complete a secondary high accuracy confirmatory test. The COBRA Kiosk incorporates three sub-systems to record a successful SERs nano-patch measurement: 1) a SERs optical scanning system; 2) an intelligent video/infrared camera with visual feedback to subject that aids self-positioning within the field of view for SERS nano-patch measurements; 3) a fast SERS processing tool to provide feedback to the subject within seconds. The intelligent camera system aids the subject in positioning the mask-embedded nano-patch so that recorded SERs data has sufficient SNR. A first step to this is establishing the SERs detection and sensitivity limitation as it relates to scanning field-of-view, depth-of-field, ambient light effects, and subject/mask nanopatch movement variations.

Benchtop Design and Prototyping of Optical Scanning and SERS Detection System.

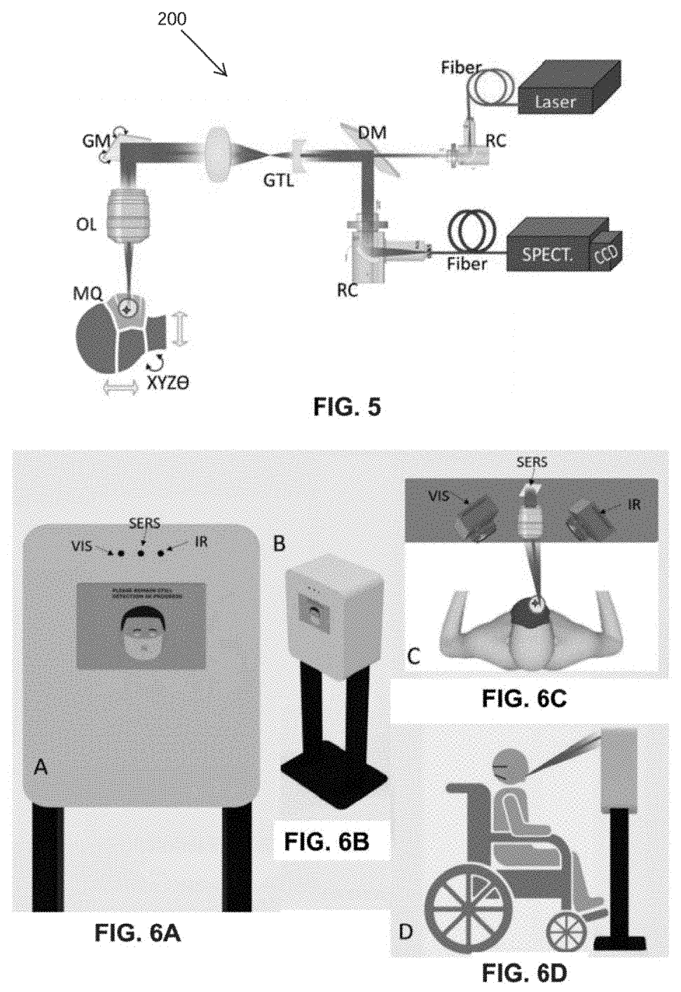

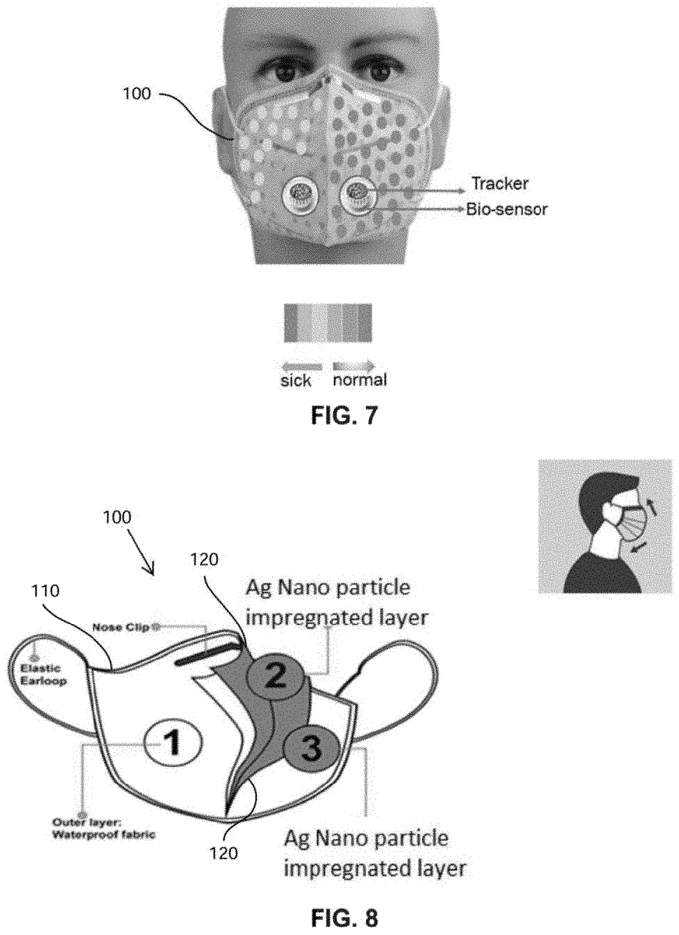

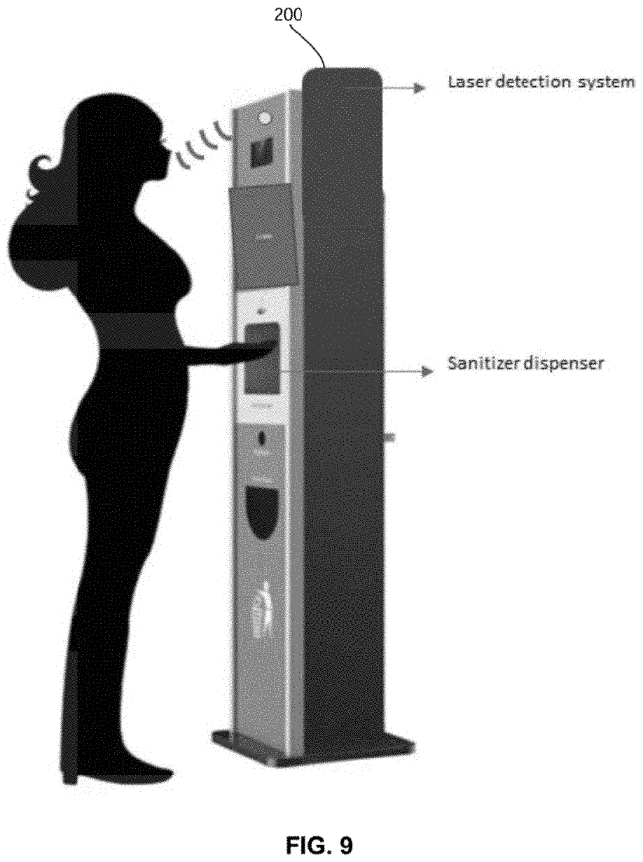

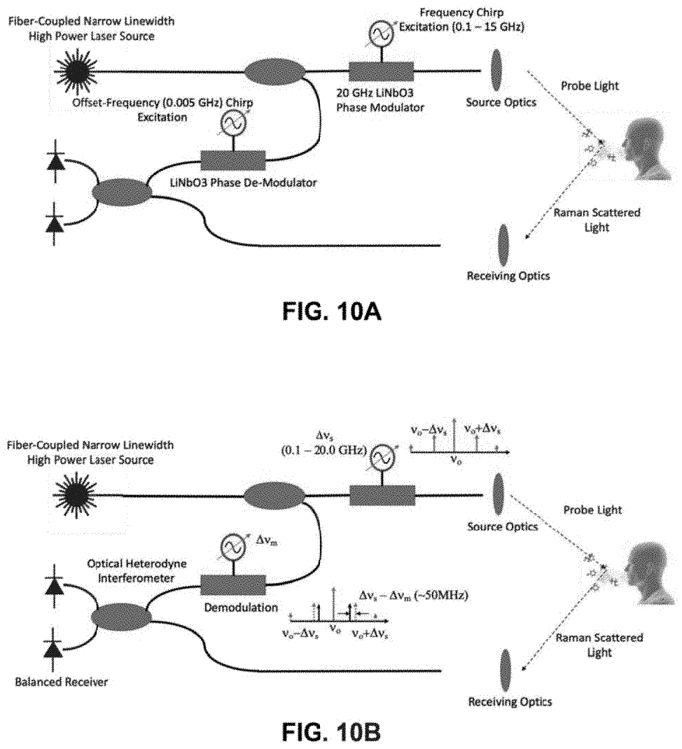

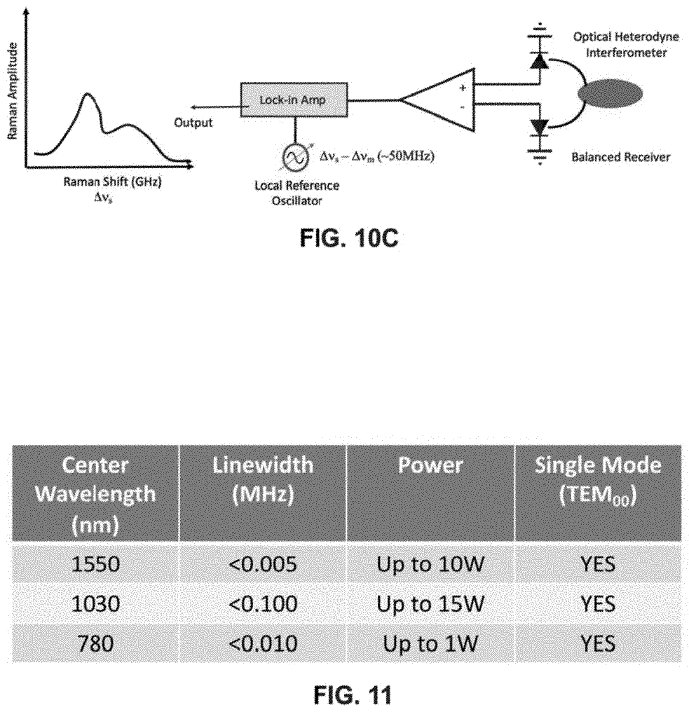

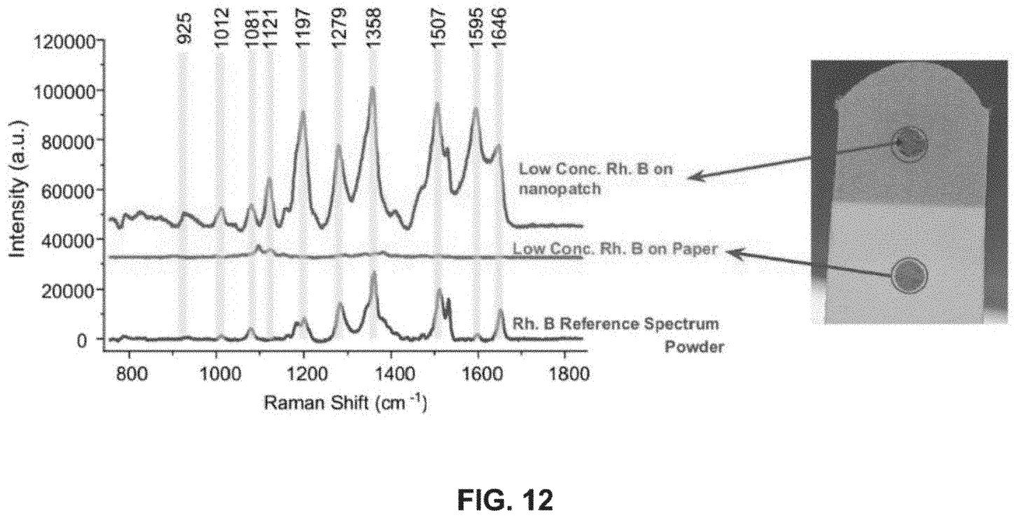

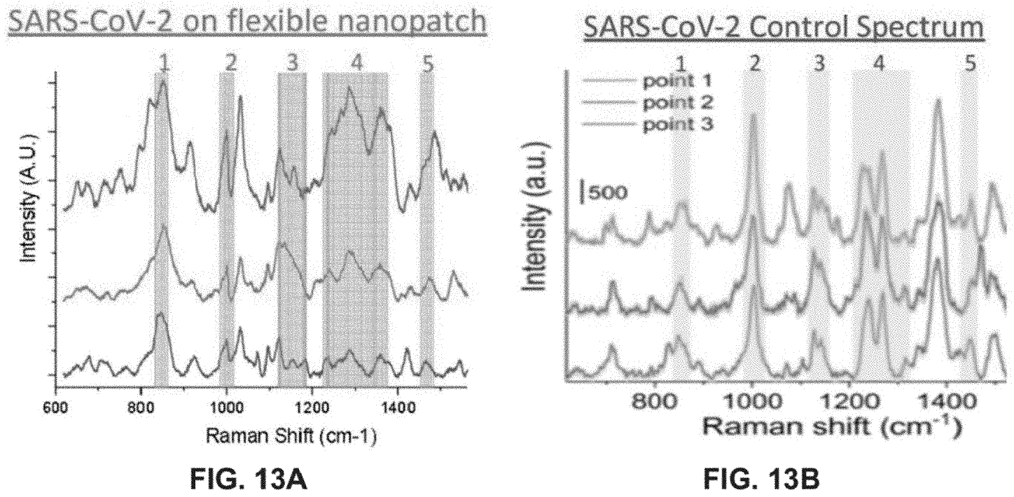

This system provides constraints on SERS sensitivity and establishes SERS nanopatch placement requirements that guide the intelligent camera system design (FIG. 5). Each time a subject walks up to the COBRA Kiosk wearing an intelligent mask and is within the field of view of the scanning system, a safe and reliable SERS measurement is recorded. The bench-top system consists of a Raman laser excitation source (a fiber-coupled single-mode diode laser, λ=785 nm.). The laser beam is collimated, expanded, and delivered to the SERS nanopatch through an f-θ scanning optical objective. After image-locking on the nanopatch fiducials recorded by the intelligent camera system, the galvanometer mirror (GM) maintains the SERS excitation beam on the mask-embedded nanopatch. The image-lock is verified before each SERS measurement and updated at about 20 Hz. The image-lock ensures the SERS excitation laser is always directed on the nanopatch regardless of subject movement. The backscattered SERS signal from the nanopatch is collected through a dichroic by a spectrograph and a deep cooling CCD camera through an optical fiber (50 μm, NA=0.22), which also acts as a pinhole. The power delivered to the nanopatch is approximately 250 mW (below the American National Standard For Safe Use Of Lasers limit ANSI Z136.1-2014 of 0.5 W for skin) with a lateral resolution FWHM (full width at half maximum) of the point spread function of approx. 50 μm with an approx. spectral resolution of the system being 8 cm⁻¹. Mitigation strategies are undertaken to ensure the laser is only ON when measurement of the SERS nanopatch is image-locked onto by the mask alignment system to prevent any stray light damage to subjects (eye or skin) or people in the vicinity of the scanning system. If ambient light degrades SNR of SERS data, black light absorbing shields are installed on the top and sides of the kiosk.

A masked light-weight dummy wearing mask-embedded SERS nano-patch is placed on X,Y,Z, tilt(θ) stage to obtain specific scanning constraints given a working distance of 30 cm that allows for sufficient SERS sensitivity namely: 1) depth of field, 2) minimum scan area, 3) beam widths of excitation/receiving irradiation at the back focal plane of objective lens, 4) minimum field flatness of the mask required for the scanning system. These parameters inform the design of the intelligent camera and image-lock guidance system to ensure the laser excitation beam is directed at the SERS nanopatch.

Stand-Alone COBRA Kiosk with Integrated Optical Scanning and SERS Detection System:

The stand-alone kiosk consists of three subsystems: 1) COBRA Kiosk optics and SERS scanning system; 2) an intelligent image-lock camera system; and 3) software for SERS detection.

1) COBRA Kiosk Optics and SERS Scanning System:

COBRA Kiosk optics has the following components: A visible camera, an optimized SERS scanning system, and an IR Camera. Optical axes of the three sub-systems are confined to a plane (FIGS. 6A-C) where the visible camera image is displayed to the user for self-alignment of the nanopatch with respect to the SERS scanning system. All three sub-systems are mounted on a tilt-panning platform mount that allows directing the SERS excitation beam onto the mask-embedded nanopatch of individuals with variable heights ranging from 1.5-2.5 meters.

2) Intelligent Image-Lock Camera System:

A computer vision image-locking system is trained using Convolutional Neural Networks to detect fiducials on the SERS nanopatch acquisition location. The mask has a high-contrast fiducial pattern at the collection site for the machine learning algorithm to lock-onto the COBRA mask. A suitable candidate pattern is a QR code, which can encode hundreds of bits of information, such as mask serial number, manufacturing dates, and a checksum code. The QR code checksum may be calculated by kiosk to confirm that the data is present and can achieve an unobstructed optical lock on the location site. To avoid ink from the QR code confounding the SERS measurement, a small inkless reticle area is positioned outside the QR code for SERS collection.

SERS acquisition and analysis are performed in real-time, with an acquisition rate of 20 Hz. Once the mask fiducial is recognized, a low power IR aiming beam is used to indicate where the SERS excitation beam intersects the mask and is used as a secondary feedback mechanism to control the galvanometer scanning system. The goal of image-locking is to ensure that when the SERS excitation beam is ON, emitted light is always interrogating the nanopatch. Galvanometer positioning of the SERS excitation laser is updated for each verified image-lock of the mask fiducials. The SERS laser emits for less than 50 ms and the processes of mask detection repeat with an image-lock until sufficient SERS data has been collected to make an analysis.

The test subject has visual feedback through a screen, with prompts about where to stand and how to position themselves in front of the COBRA kiosk. This feedback consists of visible and infrared camera images. IR images are collected by a FLIR Lepton, a small, inexpensive IR camera that may also be used as another data point (i.e. body temperature) in assessing a subject's health. The computer is an industrial Raspberry Pi 4 system with an HDMI screen to display graphics. The Raspberry Pi 4 also has connections and easy access to SPI and I2C busses that are used with the FLIR Lepton. Finally, the Raspberry Pi 4 supports a No Infrared (NoIR) camera, a Sony IMX21 8 megapixel imaging chip suitable for streaming video at 1080p resolution.

3) Software for SERS Detection:

The data collected is used to construct a machine learning algorithm. Two types of approaches are investigated, depending on the type of data that is available. The first approach is an auto-encoder, which can be trained only on “true” data, without the need for other types of classifiers. The auto-encoder output is an error, when input data looks like “true” data, the error is low. The more different input data is, the higher the error. This removes the issue of having class imbalance. As more data is collected and new classes emerge, a second approach consisting of a traditional multi-class classifier neural networks may be created with proper care taken to balance the classes during training.

Outcomes:

A stand-alone COBRA Kiosk that records an at-a-distance SERS nanopatch measurement is assembled. The intelligent camera system and image-lock ensure that no misdirection of SERS excitation laser light occurs. The SERS excitation beam is only directed and turned ON after the intelligent camera system verifies an image-lock on the mask fiducials and thereby mitigates risk of stray SERS excitation light.

Alternative Approaches:

A handheld “gun” scanner that is manually directed at a test subject may also be used. The handheld design removes the complication and constraints requiring an intelligent camera system and image-lock verification. The handheld gun scanner is placed in contact with the SERS nanopatch, flattening the nanopatch with respect to the gun optical interface, thus removing all variations possible from subject movement and simplifying the design on the SERS scanning optics, while reducing the working distance and improving SERS SNR data.

Validation for Operation of COBRA Kiosk to Screen Moving Subjects. The COBRA Kiosk screening system and decision-making algorithm are validated with robot-actuated mask-wearing dummies that simulate a range of human dimensions and kinetic movements.

Light-Weight Human Dummies Fitted with an N95 Mask-Embedded SERS Nanopatch.

Light-weight human dummies are manufactured using a large field three-dimensional polymer printing process. Custom dummies are required to achieve the lightweight goal and to represent a wide range of human features. Superior frontal body surfaces in the coronal plane (anatomically) are printed that simulate human subjects ranging over 1.5-2.5 meters in height. Lightweight superior frontal human forms are realized by balancing thickness and strength of the dummies. A mechanical mounting stand-off is printed on the backside of each form for mechanical mounting of the dummy and UR3 robotic arm. At least ten human forms are selected that represent a uniform sampling about the mean of the distribution of human heights and lateral dimensions in a target population.

An ultrasonic welding process is developed to bond SERS nanopatches to N95 masks. Ultrasonic welding is widely used to bond sub-components of N95 masks and has the advantage of providing high-strength and long-lasting bonds without introducing any extraneous non-biocompatible materials. Embedded SERS nanopatches are oriented with a nanostructured material facing against the outer surface of the N95 mask and in-line with the oral cavity. The nanostructured material collects viral particles emitted from an infected individual that escape the underlying mask material. An ultrasonic welding apparatus allows for rapid single-step bonding of SERS nanopatches to N95 masks. Five thousand N95 masks are purchased and SERS nanopatches are bonded on an as-needed basis using the ultrasonic welding apparatus.

Outcomes:

At least ten light-weight human frontal dummies are fitted with N95 masks with an embedded SERS nanopatch. Thousands of SERS nanopatch embedded N95 masks may be produced using the ultrasonic welding process.

Alternative Approaches:

Since N95 masks screen particulates 300 nm or greater, many SARS-CoV-2 viral particles may be convectively transported by exhalation onto the nanostructured surface on the SERS nanopatch. Although many viral particles are dissolved in small water droplets when emitted by the human respiratory system, evaporation allows the viral particles to be convectively transported onto the nanostructured surface. If existing N95 mask material is too thick and viral loads are insufficient, a mask-thinning process may be used. Instead of directly bonding SERS nanopatches, N95 mask material is first thinned using an erbium yttrium aluminum garnet (Er.YAG) laser (λ=2.94 μm). Er.YAG laser radiation is strongly absorbed and can controllably thin (ablate) N95 mask material. A thinning process uses optical coherence tomography to monitor mask material thickness during laser processing. The aerosolized SARS-CoV-2 simulator (FIG. 4) calibrated to validate the patches is used to determine the efficiency of virus transmission from inside the N95 mask to the outer mask surface where the SERS nanopatch is bonded as well the effectiveness of mitigation techniques such as laser thinning.

COBRA Kiosk Collection of SERS Nanopatch Data from Robot-Actuated Mask-Wearing Dummies.

Light-weight human dummies are rigidly attached to the UR3 robotic arm with a custom-designed aluminum mechanical linkage. The mechanical linkage between the UR3 robotic arm and each of the ten dummies is installed and a range of tests is completed to verify secure and safe operation over a wide range of kinematic motions.

The UR3 robot is programmed to translate dummies starting from a two-meter distance up to the COBRA Kiosk. The robot is programmed to provide random approaches to the COBRA Kiosk by varying kinematic parameters including approach direction and speed that are derived by sampling values from a selected random distribution. Once the dummy is stationary in place in front of the COBRA Kiosk, the self-guidance system is tested by inputting the error signal derived from the intelligent camera system into a corrective robotic movement. After the COBRA intelligent camera system image-locks onto SERS nanopatch fiducials, a low-frequency jitter motion is introduced to the dummy to test the SERS excitation beam aiming system. Capability of the image-lock feedback to maintain the SERS excitation beam on the nanopatch for sixty seconds is tested objectively by varying amplitude of the low-frequency dummy head jitter. SERS data is recorded after each verified image-lock. Signal-to-noise ratio (SNR) of SERS data recorded with random head movement is compared to that recorded for a static dummy. A test-base of sixty dummy kinematic movements is formulated that includes at least ten dummies, six approaches, and random low-frequency head jitter.

Outcomes:

A distribution of kinematic approaches that simulate human movement up to a COBRA Kiosk for a SARS-CoV-2 screening is established. Operation of the COBRA Kiosk image-lock system is verified to maintain safe and precise aiming of the SERS excitation beam on a randomly-jittering dummy wearing an N95 mask-embedded nanopatch for sixty seconds while recording high-quality SERS data.

Alternative Approaches:

A key risk of the COBRA Kiosk SARS-CoV-2 screening system is mis-direction of SERS excitation light. The auto image-lock feature is designed to safely maintain the SERS excitation beam on the nanopatch to record high SNR SERS data when the test subject may be moving. If frequency response of the image-lock feature is too slow for some test subjects (e.g., individuals with tremors) a higher frame rate camera is identified. A faster camera increases the frequency response of the auto image-lock feedback system beyond the existing 20 Hz and better responds to any anticipated head jitter.

Live COBRA Kiosk Screening of Normal and Infected Robot-Actuated Mask-Wearing Dummies.

To verify operation of the COBRA Kiosk screening system and the decision-making algorithm, testing on moving normal and infected subjects is completed. Positive and negative SERS nanopatches are prepared and ultrasonically bonded to N95 masks. Initially, one-thousand SERS nanopatches are prepared with equal numbers of positive and negative controls. Nanopatches are seeded using the aerosolized SARS-CoV-2 simulator (FIG. 4). N95 masks embedded with SERS nanopatches are randomly selected for a dummy and kinematic approach for COBRA Kiosk screening. SERS data for each dummy and kinematic approach is recorded over a thirty-second time period.

A decision-making algorithm for classifying acquired SERS spectra as positive or negative for SARS-CoV-2 is developed. SERS spectra are preprocessed for intensity calibration, cosmic ray and background removal, and wavenumber calibration. An array of machine learning algorithms (neural networks, support vector machine, decision tree, random forest, etc.) to classify the spectra are screened. 3-fold cross-validation is used to split the dataset into three equal groups, one group is used for the validation set and the other groups as the training set. The average accuracy of the validation is used to select the best model. Using a receiver operator characteristic (ROC) curve, the area under the curve (AUC) and specificity at a fixed sensitivity of 98% are used as accuracy performance metrics. The top-performing algorithm is selected and fixed at an operating point of high sensitivity (98%) for the following validation task.

After refinement of the decision-making algorithm using retrospectively recorded COBRA Kiosk screening data, ‘live’ testing of robot-actuated test dummies is performed. The refined decision-making algorithm is coded onto a computing platform with a real-time operating system so that the output can be obtained in less than 0.5 seconds after recording SERS data. Based on the results of retrospectively analyzed COBRA Kiosk screening data, a target SERS recording time (2 seconds) is established. Live COBRA Kiosk screening data is recorded from one-thousand dummies (50% positive, 50% negative). Ability of the COBRA Kiosk to provide accurate real-time detection of SARS-CoV-2 is objectively assessed in terms of sensitivity and specificity. Statistical sample sizes are estimated to detect a 10% precision in Se&Sp at 95% (P=0.8, α=0.05).

Outcomes:

A completed COBRA Kiosk SARS-CoV-2 system capable of screening ‘live’ subjects exhibiting a range of human kinematic motions. COBRA Kiosk screening time is six seconds and allows for a 2 second SERS measurement.

Alternative Approaches:

If the required SERS measurement time is much longer than desired, additional test subject information may be included in the decision-making algorithm. Additional information might include for example infrared image data and facial reflectance spectra that provide additional diagnostic value.

The COBRA Kiosk screening system is a highly innovative approach for high-throughput, massively-scalable and economical SARS-CoV-2 screening. The COBRA Kiosk may be deployed in airports and other transportation hubs to limit spread of infectious viral diseases by quarantining individuals seeking to travel across national boundaries. The devastating social and economic consequences many have suffered from the COVID-19 pandemic may be eliminated with successful deployment of the COBRA Kiosk screening system. The underlying SERS technology may provide a basis for rapid detection of many infectious agents and be applied to ensure individual health and wellbeing.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system for high-throughput pathogenic particle screening, the system comprising:
a. a facemask (100) for capturing and preparing pathogenic particles for screening, the facemask (100) comprising:
i. a barrier material (110), configured to allow air flow through the barrier material (110) and to at least partially block the passage of pathogenic particles through the barrier material (110); and
ii. a nanostructured material (120), configured to enhance a Raman scattering signal amplitude of the pathogenic particles;
wherein the facemask (100) is configured to be positioned over the oral and nasal cavities of a user (300) so as to capture any particles expelled by the user (300) on the nanostructured material (120), so as to prepare the particles for screening; and
b. a surface-enhanced Raman scattering (SERS) detector (200), configured to record a SERS spectrum from the facemask (100), so as to provide for high-throughput screening for the pathogenic particle.

2. The system of claim 1, wherein the pathogenic particle is a SARS-CoV-2, Coronavirus, middle east respiratory syndrome (MERS), severe acute respiratory syndrome (SARS) coronavirus, influenza, zika virus, Herpes, Zoster, Flavivirus, Redondo virus, Orthomyxovirus, Picornavirus, Papillomavirus, Syncytial virus, Adenovirus, human immunodeficiency virus (HIV), Circovirus, Anellovirus, Polyoma virus, Cytomegalovirus, Variola virus, Epstein-Barr virus, bacteria-invading virus, influenza, measles, mumps, rhinovirus, pertussis, or tuberculosis (TB) particle.

3. The system of claim 1, wherein the detector (200) includes a laser light source, configured to be directed at a portion of the facemask (100).

4. The system of claim 1, additionally comprising a microprocessor configured to classify the SERS spectrum as positive or negative for the pathogenic particle through a machine learning algorithm.

5. The system of claim 4, wherein the SERS detector (200) uses optical heterodyning for ultra-low frequency Raman spectroscopy configured for the 0.1-50 GHz range.

6. The system of claim 1, wherein the SERS detector (200) is a remote detector and the facemask (100) may be screened for the pathogenic particle at a distance.

7. The system of claim 6, wherein the second test comprises a polymerase chain reaction (PCR) test or a viral antibody test.

8. The system of claim 1, wherein the SERS detector (200) is housed in a kiosk comprising a visible-wavelength camera and an infrared wavelength camera for detecting a position of the facemask (100) in relation to the SERS detector (200).

9. The system of claim 8, wherein the kiosk is configured to provide feedback to the user (300) so as to guide placement of the facemask (100) within a field of view of the SERS detector (200).

10. The system of claim 1, wherein the detector (200) is a handheld fiber optic probe.

11. The system of claim 1, wherein use of the system does not require collection of bodily fluids or tissue material.

12. The system of claim 1, wherein the SERS detector (200) is mounted on a component of a body scanner which rotates around the user (300).

13. A facemask (100) for capturing and preparing a pathogenic particle for screening, the facemask (100) comprising:
a. a barrier material (110), configured to allow air flow through the barrier material (110) and to at least partially block the passage of pathogenic particles through the barrier material (110); and
b. a nanostructured material (120), configured to enhance a Raman scattering signal amplitude of the pathogenic particle;
wherein the facemask (100) is configured to be positioned over the oral and nasal cavities of a user (300) so as to capture any pathogenic particles expelled by the user (300) on the nanostructured material (120), so as to prepare the particle for screening.

14. The facemask (100) of claim 13, wherein the nanostructured material (120) comprises a nano-patch, a nanosurface or a dispersion of nanoparticles, nano-rods, nano-stars, nano-spheres, nano-cylinders, nano-cubes, nano-ellipsoids, nano-planar or nano-spiral-twisted particles, gold, silver, copper, aluminum, another metal, or a doped semiconductor.

15. The facemask (100) of claim 13, additionally comprising one or more CO₂ sensors.

16. The facemask (100) of claim 15, wherein the CO₂ sensors are configured to change color when the facemask (100) has been worn for a sufficient length of time for accurate screening.

17. The facemask (100) of claim 13, additionally comprising a quick response (QR) code linked to an identifier for the user (300).

18. A system for high-throughput pathogenic particle screening, the system comprising:
a. a capture device for capturing and preparing pathogenic particles for screening, the capture device comprising:
i. a nanostructured material (120), configured to enhance a Raman scattering signal amplitude of the pathogenic particles; and
ii. a support structure for supporting the nanostructured material (120);
wherein the capture device is configured to capture particles from the user (300) on the nanostructured material (120), so as to prepare the particles for screening; and
b. a surface-enhanced Raman scattering (SERS) detector (200), configured to record a SERS spectrum from the capture device, so as to provide for high-throughput screening for the pathogenic particle.

19. The system of claim 18, wherein the capture device additionally comprises a barrier material (110) configured to allow air flow through the barrier material (110) and to at least partially block the passage of pathogenic particles through the barrier material (110).

20. The system of claim 18, wherein the capture device comprises a swab, an air filter, or other test structure.
